(19) United States
(12) Patent Application Publication
(10) Pub. No.: US 2005/0107759 A1
(43) Pub. Date: May 19, 2005

(54) Absorbent article with three-dimensional extrudate forming sap containment wells

(76) Inventors: Andrew Waksmundzki, Jackson, NJ (US); John Litvay, Downingtown, PA (US); Frank Glaug, Chester Springs, PA (US)

Correspondence Address: Christopher C. Campbell, Esq.;Hunton & Williams, Suite 1200, 1900 K. Street, Washington, DC, 20006-1109, US

(21) Appl. No.: 10/951,792

(22) Filed: Sep. 29, 2004

Related U.S. Application Data
(63) Continuation-in-part of application No. 10/050,045, filed on Jan. 17, 2002.

(51) Int. Cl. A61F 13/15; A61F 13/20

(52) U.S. Cl. 604/378

(57) ABSTRACT

An absorbent core for use in an absorbent article, having a central fibrous layer having synthetic fibers, and a plurality of discrete containment wells that contain superabsorbent particles. The plurality of discrete containment wells are formed from a three-dimensional extrudate layer having a plurality of discrete voids, and a base layer that is laminated to one surface of the extrudate layer. The containment wells are provided by the voids of the extrudate layer, the perimeter being provided by the extrudate surrounding the voids, and the base of the wells being provided by the base layer. Superabsorbent particles are deposited and enclosed in the discrete containment wells, and are prevented from shifting to other portions of the absorbent core. An absorbent article including the absorbent core, and a method for providing an absorbent article including the absorbent core are also described.

This application is a continuation-in-part of U.S. application Ser. No. 10/050,045 filed Jan. 17, 2002, entitled “Absorbent Laminate,” the contents of which are incorporated herein in their entirety to the extent that it is consistent with this invention and application.

### BACKGROUND OF THE INVENTION

1. Field of the Invention

The present invention relates generally to an absorbent core for an absorbent article, and more particularly to an absorbent core having a plurality of discrete containment wells formed by a three-dimensional extrudate and a base layer. The containment wells provide for additional retention of superabsorbent particles, as well as the potential for zoned absorbency due to specific placement of superabsorbent particles in the containment wells. Such absorbent cores provide increased absorbency, additional flexibility of creating precise zoning of particular properties throughout the core, and they provide improved comfort and fit.

2. Description of Related Art

Disposable absorbent garments such as infant diapers or training pants, adult incontinence products and other such products typically were constructed with a moisture-impervious outer backing sheet, a moisture-pervious body-contacting inner liner sheet, and a moisture-absorbent core sandwiched between the liner and backing sheets. Much effort has been expended to find cost-effective materials for absorbent cores that display favorable liquid absorbency and retention. Superabsorbent materials in the form of granules, beads, fibers, bits of film, globules, etc., have been favored for such purposes. Such superabsorbent materials generally are polymeric gelling materials that are capable of absorbing and retaining even under moderate pressure large quantities of liquid, such as water and body wastes, relative to their own weight.

The superabsorbent material generally is a water-insoluble but water-swellable polymeric substance capable of absorbing water in an amount which is at least ten times the weight of the substance in its dry form. In one type of superabsorbent material, the particles or fibers may be described chemically as having a back bone of natural or synthetic polymers with hydrophilic groups or polymers containing hydrophilic groups being chemically bonded to the back bone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated or phosphorylated, causing the SAP to be highly hydrophilic. Such modified polymers may also be cross-linked to reduce their water-solubility.

The ability of a superabsorbent material to absorb liquid typically is dependent upon the form, position, and/or manner in which particles of the superabsorbent are incorporated into the absorbent core. Whenever a particle of the superabsorbent material and absorbent core is wetted, it swells and forms a gel. Gel formation can block liquid transmission into the interior of the absorbent core, a phenomenon called “gel blocking.” Gel blocking prevents liquid from rapidly diffusing or wicking past the “blocking” particles of superabsorbent (e.g., those particles that have swelled and touched an adjacent swelled particle), causing portions of a partially hydrated core to become inaccessible to multiple doses of urine. Further absorption of liquid by the absorbent core must then take place via a diffusion process. This is typically much slower than the rate at which liquid is applied to the core. Gel blocking often leads to leakage from the absorbent article well before all of the absorbent material in the core is fully saturated.

Despite the incidence of gel blocking, superabsorbent materials are commonly incorporated into absorbent cores because they absorb and retain large quantities of liquid, even under load. However, in order for superabsorbent materials to function, the liquid being absorbed in the absorbent structure must be transported to unsaturated superabsorbent material. In other words, the superabsorbent material must be placed in a position to be contacted by liquid. Furthermore, as the superabsorbent material absorbs the liquid it must be allowed to swell. If the superabsorbent material is prevented from swelling, it will cease absorbing liquids.

Adequate absorbency of liquid by the absorbent core at the point of initial liquid contact and rapid distribution of liquid away from this point is necessary to ensure that the absorbent core has sufficient capacity to absorb subsequently deposited liquids. Previously known absorbent cores have thus attempted. to absorb quickly and distribute large quantities of liquids throughout the absorbent core while minimizing gel blocking during absorption of multiple doses of liquid.

In general, some of the important performance attributes of an absorbent core of a diaper (or any other absorbent garment) are functional capacity, rate of absorption, core stability in use, type of SAP, ratio of fibrous material to SAP, the type and basis weight of glue or tackifying agent used to adhere the SAP to the fibrous material or tissue wrapping, and the basis weight of the core. Absorption under load or AUL is a good measure of functional capacity and the rate at which that absorption occurs. AUL is believed to be a function of both SAP basis weight (mass per unit area) and the composition of SAP used in the composite. Increasing the basis weight decreases the performance/cost ratio of the absorbent core, making them uneconomical. Also, increased basis weights tend to affect the fit and comfort of the garment, as well as impacting the packaging and shipping costs.

It is known to provide absorbent laminates comprised of, for example, an upper and lower layers, and a central fibrous layer containing from 50% to 95% by weight SAP. U.S. Pat. No. 6,068,620, the disclosure of which is incorporated herein by reference in its entirety, discloses that the upper and lower layers are comprised of tissue, airlaid fluff pulp or synthetic non-woven fibrous layers. The upper and lower layers are said to assist in maintaining the integrity of the core, the laminate layered arrangement is said to minimize gel blocking, and the laminate can be folded in various configurations.

It also is known to provide absorbent cores comprised of differing materials in an attempt to maximize comfort and efficiency of the core, and to provide areas having varying degrees of absorbency. U.S. Pat. No. 5,849,002, the disclosure of which is incorporated by reference herein in its entirety, discloses absorbent cores having three zones: (i) one zone for receiving fluids; (ii) one zone for distributing and storing fluids; and (iii) one zone for preventing leakage. U.S. Pat. No. 5,853,402, the disclosure of which is incorporated by reference herein in its entirety, discloses composite absorbent cores comprising at least an absorbent material and a porous resilient material. Other composite, zoned, or multi-component cores are disclosed in, for example, U.S. Pat. No. 5,681,300 (blended absorbent core), U.S. Pat. No. 5,882,464 (crimping to join two absorbent structures), U.S. Pat. No. 5,891,120 (varying SAP concentration throughout core), U.S. Pat. Nos. 5,425,725 and 5,983,650 (multiple fiber free SAP pockets in core), and U.S. Pat. No. 5,922,165 (method of joining outer layers with absorbent core disposed between the outer layers). The respective disclosures of each of these documents are incorporated by reference herein in their entirety.

It is also known to attach a cover sheet and a backing sheet to form pockets in which the a fluid absorbent material is stored. U.S. Pat. No. 4,360,021, the disclosure of which is incorporated by reference herein in its entirety, discloses an absorbent article in which fluid absorbent material is deposited in portions of the backing sheet, and the cover sheet is placed over the absorbent material, and pressed towards the exposed parts of the backing sheet to cause bonding of the backing sheet and the cover sheet to form pockets in which the absorbent material is stored. U.S. Pat. Nos. 5,643,238 and 5,863,288, the disclosures of which are incorporated by reference herein in their entirety, disclose absorbent cores comprising storage cells and acquisition cells. Within the storage cells of the absorbent core is disposed a quantity of superabsorbent material, while the acquisition cells are devoid of superabsorbent material or other materials that would impede liquid movement therethrough.

The disclosure herein of disadvantages and poor performance of known products, methods, and apparatus is not intended to limit the scope of the. present invention. Indeed, various embodiments of the invention may include some of the known products, methods, and apparatus without suffering from the disadvantages.

### SUMMARY OF THE INVENTION

It would be desirable to provide an absorbent garment having an improved ability to retain fluids and consequently, to prevent leakage.. It also would be desirable to provide an absorbent core that includes an increased amount of superabsorbent polymers, but at the same time does not suffer from gel blocking to an appreciable extent. A further desirable feature would be to provide an absorbent core having varying areas of absorbency to account for variations in gender and age, that is relatively easy and inexpensive to manufacture.

It therefore is a feature of an embodiment of the invention to provide an absorbent garment having an improved ability to retain fluids, especially in areas of the core where fluid retention is needed most. It is an additional feature of an embodiment of the invention to provide an absorbent garment that includes an absorbent core having SAP particles as a substantial percentage of its basis weight, but at the same time reducing gel blocking, i.e., retaining high SAP efficiency. An additional feature of the invention is to provide an absorbent article having specific desired properties in select areas of the absorbent core that is relatively inexpensive to manufacture, that provides the improved properties above, and that is comfortable to wear.

These and other features of the invention can be achieved by an absorbent article including a top sheet, a back sheet and an absorbent core disposed between the top sheet and the back sheet. The absorbent core of the invention preferably is comprised of a central fibrous layer comprising synthetic tow fibers, a three-dimensional extrudate having a plurality of voids, a base layer, and superabsorbent polymer. Preferably, the three-dimensional extrudate layer and the base layer form a plurality of discrete containment wells in which at least some of the superabsorbent polymer is contained.

In accordance with an additional embodiment of the invention, there is provided a method of making an absorbent article that includes providing a top sheet material and a back sheet material. The method also includes preparing an absorbent core that includes providing a central fibrous layer that has synthetic tow fibers, providing a three-dimensional extrudate layer having a plurality of voids and providing a base layer. The method further includes disposing the base layer adjacent the three-dimensional extrudate layer, thereby forming a plurality of discrete containment wells, and providing superabsorbent particles to the containment wells. The central fibrous layer is preferably disposed above the three-dimensional extrudate layer and the base layer in the absorbent core. The absorbent core is then disposed between the top sheet material and the back sheet material.

These and other features and advantages of the preferred embodiments will become more readily apparent when the detailed description of the preferred embodiments is read in conjunction with the attached drawings.

### BRIEF DESCRIPTION OF THE DRAWINGS

FIG. 1 is a partially cut-away view of an embodiment of the present invention, shown with top sheet facing down and the elastic members fully stretched in the main portion of the garment;

FIG. 2 is a cross-sectional view of the absorbent garment in FIG. 1, taken along line 2-2;

FIG. 3a is a top view of a three-dimensional extrudate layer in accordance with one embodiment of the invention;

FIG. 3b is a top view of a three-dimensional extrudate layer in accordance with one embodiment of the invention;

FIG. 3c is a top view of a three-dimensional extrudate layer in accordance with one embodiment of the invention;

FIG. 3d is a top view of a three-dimensional extrudate layer in accordance with one embodiment of the invention;

FIG. 3e is a top view of a three-dimensional extrudate layer in accordance with one embodiment of the invention;

FIG. 4a is a cross-sectional view of an absorbent core in accordance with an embodiment of the invention;

FIG. 4b is a cross-sectional view of an absorbent core in accordance with an embodiment of the invention;

FIG. 4c is a cross-sectional view of an absorbent core in accordance with an embodiment of the invention;

FIG. 4d is a cross-sectional view of an absorbent core in accordance with an embodiment of the invention;

FIG. 4e is a cross-sectional view of an absorbent core in accordance with an embodiment of the invention;

FIG. 4f is a cross-sectional view of an absorbent core in accordance with an embodiment of the invention;

FIG. 5 is a cross-sectional view of containment wells containing superabsorbent polymer, in accordance with an embodiment of the invention;

FIG. 6 is an illustration of an apparatus useful in carrying out a method of making an absorbent garment in accordance with the present invention;

FIG. 7 is an illustration of an apparatus useful in carrying out a method of making an absorbent garment in accordance with the present invention; and

FIG. 8 is an illustration of an apparatus useful in carrying out a method of making an absorbent garment in accordance with the present invention.

### DETAILED DESCRIPTION OF PREFERRED EMBODIMENTS

As used herein, the terms “absorbent garment,” “absorbent article” or simply “article” or “garment” refer to devices that absorb and contain body fluids and other body exudates. More specifically, these terms refer to garments that are placed against or in proximity to the body of a wearer to absorb and contain the various exudates discharged from the body. A non-exhaustive list of examples of absorbent garments includes diapers, diaper covers, disposable diapers, training pants, feminine hygiene products and adult incontinence products. Such garments may be intended to be discarded or partially discarded after a single use (“disposable” garments). Such garments may comprise essentially a single inseparable structure (“unitary” garments), or they may comprise replaceable inserts or other interchangeable parts.

The present invention may be used with all of the foregoing classes of absorbent garments, without limitation, whether disposable or otherwise. The embodiments described herein provide, as an exemplary structure, a diaper for an infant, however this is not intended to limit the claimed invention. The invention will be understood to encompass, without limitation, all classes and types of absorbent garments, including those described herein. Preferably, the absorbent core is thin in order to improve the comfort and appearance of a garment.

Throughout this description, the expressions “upper layer,” “lower layer,” “above” and “below,” which refer to the various components included in the absorbent core units of the invention (including the layers surrounding the absorbent core units) are used merely to describe the spatial relationship between the respective components. The upper layer or component “above” the other component need not always remain vertically above the core or component, and the lower layer or component “below” the other component need not always remain vertically below the core or component. Indeed, embodiments of the invention include various configurations whereby the core is folded in such a manner that the upper layer ultimately becomes the vertically highest and vertically lowest layer at the same time. Other configurations are contemplated within the context of the present invention.

The term “component” can refer, but is not limited, to designated selected regions, such as edges, corners, sides or the like; structural members, such as elastic strips, absorbent pads, stretchable layers or panels, layers of material, or the like; or a graphic. The term “graphic” can refer, but is not limited, to any design, pattern, indicia or the like.

Throughout this description, the term “disposed” and the expressions “disposed on,” “disposing on,” “disposed in,” “disposed between” and variations thereof (e.g., a description of the article being “disposed” is interposed between the words “disposed” and “on”) are intended to mean that one element can be integral with another element, or that one element can be a separate structure bonded to or placed with or placed near another element. Thus, a component that is “disposed on” an element of the absorbent garment can be formed or applied directly or indirectly to a surface of the element, formed or applied between layers of a multiple layer element, formed or applied to a substrate that is placed with or near the element, formed or applied within a layer of the element or another substrate, or other variations or combinations thereof.

Throughout this description, the terms “top sheet” and “back sheet” denote the relationship of these materials or layers with respect to the absorbent core. It is understood that additional layers may be present between the absorbent core and the top sheet and back sheet, and that additional layers and other materials may be present on the side opposite the absorbent core from either the top sheet or the back sheet.

Throughout this description, the expression “tow fibers” relates in general to any substantially continuous fiber. Tow fibers typically are used in the manufacture of staple fibers, and preferably are comprised of natural and/or synthetic thermoplastic polymers. Usually, numerous filaments are produced by melt extrusion of the molten polymer through a multi-orifice spinneret during manufacture of staple fibers from synthetic thermoplastic polymers in order that reasonably high productivity may be achieved. The groups of filaments from a plurality of spinnerets typically are combined into a tow which is then subjected to a drawing operation to impart the desired physical properties to the filaments comprising the tow. Tow as used in the context of the present invention also encompasses modified tow fibers that have been either surface or internally modified (chemically or otherwise) to improve various desired properties of the fibers (e.g., wicking, etc.).

The present invention relates generally to absorbent articles, and in particular to an absorbent article that contains a top sheet, a back sheet, and an absorbent core disposed at least partially between the top sheet and the back sheet. The absorbent core of the invention preferably has a central fibrous layer comprised of tow fiber, and a plurality of discrete containment wells that contain superabsorbent polymer (SAP) particles. Preferably, the containment wells are formed from a three-dimensional extrudate layer having a plurality of discrete void areas, and a base layer attached to one surface of the extrudate layer. The containment areas are formed from the void areas, with the walls of each well comprising the extrudate surrounding the void area, and the base of each well comprising the base layer. After the SAP is deposited in the discrete containment wells, another layer is preferably laminated to the open surface of the extrudate layer, which encloses the SAP particles within the containment wells. The discrete containment wells therefore prevent the SAP particles from shifting to other portions of the absorbent core.

The invention also relates in general to a method of making an absorbent article that includes providing a top sheet material and a back sheet material. The method also includes preparing an absorbent core that contains a three-dimensional extrudate layer, a base layer, and a central fibrous layer comprised of tow fiber. When combined, the layers form containment wells for the containment of SAP particles within the absorbent core.

Preparing the absorbent core includes forming the three-dimensional extrudate layer, and combining it with a base layer so that the two layers are in intimate contact to form a plurality of containment wells. The method further includes depositing SAP particles within the containment wells, and then laminating another layer on the open side of the three-dimensional extrudate layer, to fully enclose the deposited SAP particles within the containment wells. The layer laminated to the open side of the three-dimensional extrudate layer may be a central fibrous layer, another base layer, or another layer, such as an additional layer. The method optionally includes enclosing the absorbent core between upper and lower layers, such as tissue layers, to enclose and contain the absorbent materials. In one embodiment of the invention, the SAP particles are distributed so that the target absorbency zone has a higher concentration of SAP than other areas of the absorbent core.

The absorbent article of the invention preferably has a front waist region, a rear waist region and a crotch region positioned between the front and rear waist regions. The front waist region and rear waist region can be associated with one another to form a waist opening, and two leg openings. Those skilled in the art recognize that “front” and “rear” in the context of the invention denote for clarity purposes only the front and rear of a user, and that the absorbent article could be reversed whereby the previously described “front” portion becomes the rear portion, and vice versa.

Leg elastics preferably are provided along the leg openings for securely holding the leg openings against the thighs of the wearer to improve containment and fit. A fastening system, either resealable or permanent, preferably holds the absorbent article around the wearer's waist. The fastening system assists in associating the front waist region with the rear waist region. A pair of stand-up leg gathers or waist containment flaps may be attached to or formed from the body's side surface of the top sheet.

The preferred embodiments of the absorbent article of the invention include an absorbent core comprising both tow fibers and SAP. Within the absorbent core, the SAP particles are enclosed in a plurality of discrete containment wells formed in part by a three-dimensional extrudate layer. The absorbent core and/or the absorbent article also may include one or more additional components, such as at least one layer selected from an acquisition layer, a distribution layer, an additional fibrous layer containing SAP, a wicking layer, a storage layer, or combinations and fragments of these layers.

Other non-SAP-containing roll good materials such as latex or thermally bonded airlaid fluff pulp, (e.g., roll good available from Walkisoft, Merfin or Fort James), or synthetic spunbonded, carded, or hydro-entangled non-woven may be positioned above and below the absorbent core. The absorbent core also may be comprised of more than one absorbent core unit. The absorbent core of the invention preferably contains 50-95% by weight particulate or fibrous SAP and a tow fiber, which preferably is capable of maintaining high SAP efficiency. As described in U.S. Pat. No. 6,068,620, SAP efficiency can be expressed as the ratio of the actual SAP absorbency under load, or AUL (expressed as grams of saline absorbed per gram of SAP in the laminate), and the maximum SAP AUL obtained under ideal conditions of low basis weight where gel blocking does not occur. SAP concentrations of 50-95% provide thinner roll good composites for efficient shaping and handling. High SAP concentrations also provide thinner absorbent cores that can provide new options for product design. The absorbent core useful in the invention can be made using either a wet or dry process, but a dry process is particularly preferred.

The outer layers of the absorbent cores of the invention typically are designed for optimal wet/dry strength, liquid acquisition and distribution, as well as SAP containment. The inner layers of absorbent cores generally are designed for optimal absorbency and SAP efficiency. Designers of absorbent cores in the past have had to combine the attributes of the outer and inner layers into a homogeneous composite, often leading to an unacceptable compromise.

Absorbent cores made of fibrous materials, e.g., tow fibers, and SAP typically suffer from the inability to contain SAP in predetermined locations and prevent it from shifting to other portions of the absorbent core. These cores typically include a tackifying agent or other type of material to adhere the SAP to the fibers, or to contain the SAP. Use of tackifying agents and/or adhesives to adhere the SAP to the fibers, however, can have an adverse effect on the absorbency properties of the SAP, and can cause excessive gel blocking. Traditional cores also make it difficult to vary the absorbency throughout the cross-section of the absorbent core. These conventional cores typically were designed with a single basis weight, a single type of SAP, a single ratio of fiber to SAP, a single glue basis weight, and a single glue type. Varying any of these parameters throughout the length and/or width of the absorbent core is not practical from a manufacturing standpoint.

The present invention is premised in part on the discovery that a three-dimensional extrudate can be used to provide a plurality of containment wells in which SAP particles can be contained. The containment wells are formed by a three dimensional extrudate layer that has a plurality of voids, a base layer which is in intimate contact with one surface of the extrudate layer, and another layer which is laminated to the other surface of the extrudate layer. The discrete containment wells are useful for precise distribution of SAP to selected portions of an absorbent core, because they provide a network of discrete zones to which varying amounts of SAP can be delivered and contained in the absorbent core. In addition, the discrete containment wells prevent the SAP particles from shifting to other portions of the diaper. Because the SAP particles are completely enclosed in the void spaces of the extrudate layer—between the base layer and the other layer—they are prevented from migrating or shifting to other parts of the diaper.

The invention now will be described with reference to the attached drawings illustrating preferred embodiments of the invention. For clarity, features that appear in more than one Figure have the same reference number in each Figure.

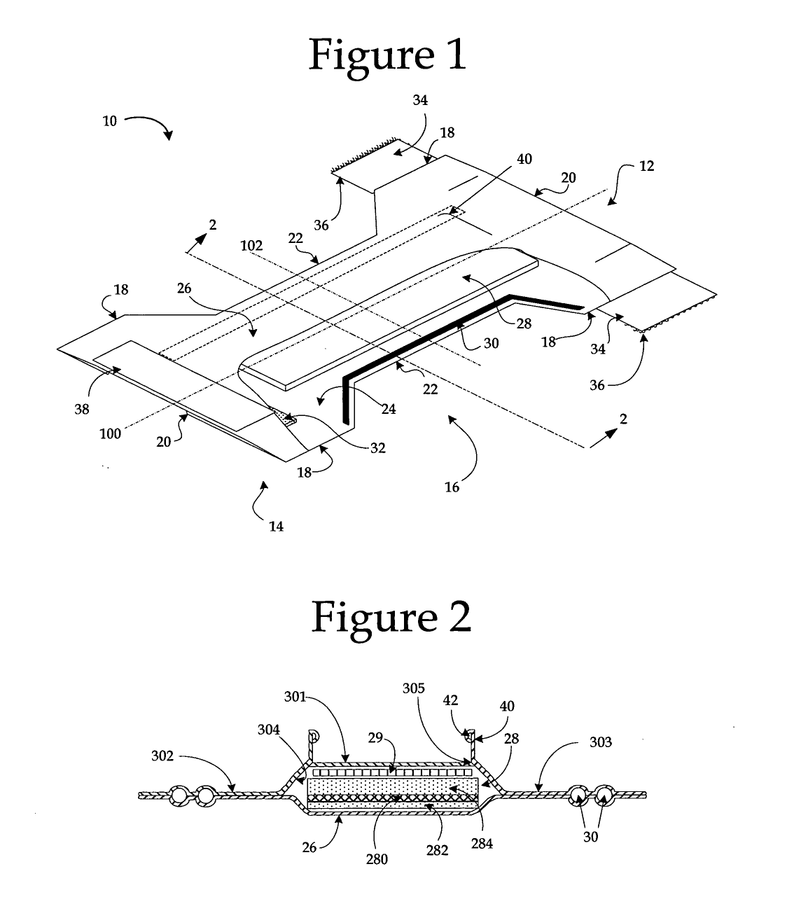

FIG. 1 is a partially cut away depiction of an exemplary embodiment of an absorbent garment 10 (preferably a disposable absorbent garment) of the present invention. The embodiment shown in FIG. 1 is an infant's diaper, however, this depiction is not intended to limit the invention, and those skilled in the art appreciate that the invention covers other types of absorbent articles. For simplicity, however, the invention will be described with reference to an infant's diaper. The garment 10 of FIG. 1 is depicted in a generally flattened position, with the body-facing side facing down, and with the various elastic components depicted in their relaxed condition with the effects of the elastics removed for clarity (when relaxed, the elastics typically cause the surrounding material to gather or “shirr”). In the flattened position, the garment 10 may have a generally hourglass shaped structure, but it may also have any other shape suitable for the given application, such as a rectangular shape, a trapezoidal shape, a “T” shape, and the like.

As used herein, the longitudinal axis 100 of the garment is the dimension of the garment corresponding to the front-to-rear dimension of the user, and the lateral (or transverse) axis 102 of the garment is the dimension corresponding to the side-to-side dimension of the user.

In use, the invention comprises a garment 10 having a pant-like configuration with a waist-encircling region and a crotch region. The waist-encircling region may comprise a first waist region 12, disposed adjacent to, for example, the back waist region of a wearer's body, and a second waist region 14, disposed adjacent to, for example, the front waist region of a wearer's body. The first and second waist regions 12, 14, may correspond to the front and back of the wearer's body, respectively, depending on whether garment 10 is attached in front of or behind the subject wearer. The first and second waist regions are joined together at or near their lateral edges 18, causing the longitudinally distal edges 20 of the garment 10 to form the perimeter of a waist opening. A crotch region 16 extends between the first and second waist regions 12, 14, and the crotch edges 22 form the perimeter of a pair of leg openings, when the garment 10 is placed on a subject wearer.

The garment 10 preferably comprises a top sheet 24, and a back sheet 26, which may be substantially coterminous with the top sheet 24. When the garment 10 is being worn, the top sheet 24 faces the wearer's body, and the back sheet 26 faces away from the wearer. An absorbent core 28 preferably is disposed between at least a portion of the top sheet 24 the back sheet 26.

An embodiment of the present invention may further comprise various additional features. One or more pairs of elastic gathers 30 (leg elastics) may extend adjacent the crotch edges 22. The garment 10 may also comprise one or more waste containment systems, such as inboard standing leg gathers 40, which preferably extend from the second waist region 14 to the first waist region 12 along opposite sides of longitudinal center line 100 (only one standing leg gather system 40 is shown in FIG. 1 for purposes of clarity). One or both of the first and second waist regions 12, 14 may also be equipped with strips of waist elastic material 32, such as elastic waist foam or other elastically extensible material, which help contract the garment around the wearer's waist, providing improved fit and leakage prevention.

The absorbent garment 10 also preferably includes fastening elements to enable attachment of the first waist region 12 to second waist region 14. Fastening elements preferably include a pair of tabs 34 that extend laterally away from opposite lateral edges 18 of the first waist region 12 of the garment 10. The tabs 34 may comprise an elastically extensible material (not shown), and may be designed to stretch around a wearer's waist to provide improved fit, comfort, and leakage protection. Such elasticized tabs 34 may be used in conjunction with, or in lieu of, waist elastic material 32, such as foam, or other elastically extensible materials.

At least one fastening mechanism 36 (collectively referred to as “fastener 36”) is attached to each tab 34 for attaching the tab to the second waist region 14, thereby providing the garment 10 with a pant-like shape, and enabling garment 10 to be fixed or otherwise fitted on the wearer. The fasteners 36 may attach to one or more target devices 38 located in the second waist region 14. For example, in one embodiment of the invention, the fastening mechanism is a hook and loop fastener, where one fastening element is a hook portion, and a corresponding target device is a loop portion of the hook and loop fastener. In another embodiment, the fastening system is a tape fastener system, where one fastening element is an adhesive tape, and a corresponding target device is a tape receiving surface. Other fastening systems may be used in this invention, as long as they are capable of fastening the garment 10 about the wearer.

Although not shown in the drawings, the absorbent garment 10 may also include grips attached along the distal edges of each tab 34 to enable a caregiver to pull the grips, and not on the ends of the tabs 34, around the wearer and over the target devices 38 to thereby secure the fasteners 36 to the one or more target devices 38.

The various parts of the garment 10 can be attached to one another or associated with one another to form a structure that preferably maintains its shape during the useful life of the garment 10. As used herein, the terms “attached,” “joined,” “associated,” and similar terms encompass configurations whereby a first part is directly joined to a second part by affixing the first part directly to the second part, by indirectly joining the first part to the second part through intermediate members, and by fixing the relative positions of various parts by capturing parts between other parts. Those skilled in the art will appreciate that various methods or combinations of methods may be used to securely join the respective parts of the garment 10 to one another.

The top sheet 24 and back sheet 26 may be constructed from a wide variety of materials known in the art. The invention is not intended to be limited to any specific materials for these components. The top sheet 24 and back sheet 26 can be shaped and sized according to the requirements of each of the various types of absorbent garment, or to accommodate various user sizes. In an embodiment of the invention in which the garment 10 is a diaper or an adult incontinence brief, the combination of top sheet 24 and back sheet 26, may have an hourglass shape, as seen in FIG. 1, or may have a rectangular, trapezoidal, “T” shape, or other shape.

Due to the wide variety of backing and liner sheet construction and materials currently available, the invention is not intended to be limited to any specific materials or constructions of these components. The back sheet 26 preferably is made from any suitable pliable liquid-impervious material known in the art. Typical back sheet materials include films of polyethylene, polypropylene, polyester, nylon, and polyvinyl chloride and blends of these materials. For example, the back sheet can be made of a polyethylene film having a thickness in the range of 0.02-0.04 mm. The back sheet 26 may be pigmented with, for example, titanium dioxide, to provide the garment 10 with a pleasing color or to render the back sheet 26 opaque enough that exudates being contained by the garment 10 are not visible from outside the garment. In addition, the back sheet 26 may be formed in such a manner that it is opaque, for example, by using various inert components in the polymeric film and then biaxially stretching the film. Other back sheet materials will be readily apparent to those skilled in the art. The back sheet 26 preferably has sufficient liquid imperviousness to prevent any leakage of fluids. The required level of liquid imperviousness may vary between different locations on the garment 10.

The back sheet 26 may further comprise separate regions having different properties. In a preferred embodiment, portions of the back sheet 26 are air-permeable to improve the breathability, and therefore comfort, of the garment 10. The different regions may be formed by making the back sheet 26 a composite of different sheet materials, chemical treatment, heat treatment, or other processes or methods known in the art. Some regions of the back sheet 26 may be fluid pervious. In one embodiment of the invention, the back sheet 26 is fluid impervious in the crotch 16, but is fluid pervious in portions of the first. and second waist regions 12, 14. The back sheet 26 may also be made from a laminate of overlaid sheets of material.

The moisture-pervious top sheet 24 can be comprised of any suitable relatively liquid-pervious material known in the art that permits passage of liquid there through. Non-woven liner sheet materials are exemplary because such materials readily allow the passage of liquids to the underlying absorbent core 28. Examples of suitable liner sheet materials include non-woven spun bond or carded webs of polypropylene, polyethylene, nylon, polyester and blends of these materials.

The back sheet 26 may be covered with a fibrous, non woven fabric such as is disclosed, for example, in U.S. Pat. 4,646,362 issued to Heran et al., the disclosure of which is hereby incorporated by reference in its entirety and in a manner consistent with this disclosure. Materials for such a fibrous outer liner include a spun-bonded non woven web of synthetic fibers such as polypropylene, polyethylene or polyester fibers; a non woven web of cellulosic fibers, textile fibers such as rayon fibers, cotton and the like, or a blend of cellulosic and textile fibers; a spun-bonded non woven web of synthetic fibers such as polypropylene; polyethylene or polyester fibers mixed with cellulosic, pulp fibers, or textile fibers; or melt blown thermoplastic fibers, such as macro fibers or micro fibers of polypropylene, polyethylene, polyester or other thermoplastic materials or mixtures of such thermoplastic macro fibers or micro fibers with cellulosic, pulp or textile fibers. Alternatively, the back sheet 26 may comprise three panels wherein a central poly back sheet panel is positioned closest to absorbent core 28 while outboard non-woven breathable side back sheet panels are attached to the side edges of the central poly back sheet panel. Alternatively, the back sheet 26 may be formed from microporous poly coverstock for added breathability.

The top sheet 24 also may be formed of three separate portions or panels. Those skilled in the art will recognize, however, that top sheet 24 need not be made of three separate panels, and that it may be comprised of one unitary item. As illustrated in more detail in FIG. 2, a first top sheet panel may comprise a central top sheet panel 301 formed from preferably a liquid-pervious material that is either hydrophobic or hydrophilic. The central top sheet panel 301 preferably extends from substantially the second waist region 14 to the first waist region 12, or a portion thereof. The second and third top sheet panels 302, 303 (e.g., outer top sheet panels), in this alternative embodiment may be positioned laterally outside of the central top sheet panel 301. The outer top sheet panels 302, 303, preferably are substantially liquid-impervious and hydrophobic, preferably at least in the crotch area. The outer edges of the outer top sheet panels may substantially follow the corresponding outer perimeter of the back sheet 26. The material for the outer top sheet portions or panels is preferably polypropylene and can be woven, non-woven, spunbonded, carded or the like, depending on the application.

The central top sheet panel may be made from any number of materials, including synthetic fibers (e.g., polypropylene or polyester fibers), natural fibers (e.g., wood or cellulose), apertured plastic films, reticulated foams and porous foams to name a few. One preferred material for a central top sheet panel is a cover stock of single ply non-woven material which may be made of carded fibers, either adhesively or thermally bonded, perforated plastic film, spun bonded fibers, or water entangled fibers, which generally weigh from 0.3-0.7 oz./sq. yd. and have appropriate and effective machine direction and cross-machine direction strength suitable for use as a baby diaper cover stock material.

The inner edges 304 (FIG. 2) of the outer top sheet portions or panels 302, 303, preferably are attached by, e.g., an adhesive, to the outer edges 305 of the inner top sheet portion or panel 301. At the point of connection with the outer edges 305 of the inner top sheet portion 301, the inner edges 304 of the outer top sheet portions 302, 303 extend upwardly to form waste containment flaps 40 (or “standing leg gathers”). The waste containment flaps 40 preferably are formed of the same material as the outer top sheet portions 302, 303, as in the embodiment shown. They are preferably an extension of the outer top sheet portions or panels 302, 303.

The standing leg gather(s) 40 preferably are disposed such that they extend laterally away from the surface of top sheet 24. Standing leg gather(s) 40 may be treated with a suitable surfactant to modify their hydrophobicity/hydrophilicity as desired, and they may be treated with skin wellness ingredients to reduce skin irritation. Alternatively, the standing leg gather(s) 40 may be formed as separate elements and then attached to the body side liner. The standing leg gather(s) 40 preferably include a portion that folds over onto itself to form a small enclosure. At least one, and depending on the size of the enclosure sometimes more than one, elastic member may be secured in the enclosure in a stretched condition. As is known in the art, when the flap elastic member 42 attempts to assume the relaxed, unstretched condition, the standing leg gather(s) 40 rise above the surface of the central top sheet portion or panel 301.

The top sheet 24 (as well as top sheet portions 301, 302, 303) may be made of any suitable relatively liquid-pervious material currently known in the art or later discovered that permits passage of a liquid there through. Examples of suitable top sheet materials include non woven spun-bonded or carded webs of polypropylene, polyethylene, nylon, polyester and blends of these materials, perforated, apertured, or reticulated films, and the like. Non woven materials are exemplary because such materials readily allow the passage of liquids to the underlying absorbent core 28. The top sheet 24 preferably comprises a single-ply non woven material that may be made of carded fibers, either adhesively or thermally bonded, spun- bonded fibers, or water entangled fibers, which generally weigh from 0.3-0.7 oz./sq. yd. and have appropriate and effective machine direction (longitudinal) and cross-machine (lateral) direction strength suitable for use as a top sheet material for the given application. The present invention is not intended to be limited to any particular material for the top sheet 24, and other top sheet materials will be readily apparent to those skilled in the art.

The top sheet 24 may further comprise several regions having different properties. In one embodiment of the present invention, the laterally distal portions of the top sheet 24, especially those used to make second and third top sheet panels 302, 303, preferably are substantially fluid impervious and hydrophobic, while the remainder of the top sheet 24 (e.g., central top sheet panel 301) is hydrophilic and fluid pervious. Different top sheet properties, such as fluid perviousness and hydrophobicity, may be imparted upon the top sheet 24 by treating the top sheet 24 with adhesives, surfactants, or other chemicals, using a composite of different materials, or by other means. The top sheet 24 may also be made from a laminate of overlaid sheets of material. The top sheet 24 also may be treated in specific areas like the crotch region, with skin wellness ingredients such as aloe, vitamin E, and the like.

As noted elsewhere herein, the top sheet 24 and back sheet 26 may be substantially coterminous, or they may have different shapes and sizes. The particular design of the top sheet 24 and back sheet 26 may be dictated by manufacturing considerations, cost considerations, and performance considerations. Preferably, the top sheet 24 is large enough to completely cover the absorbent core 28, and the back sheet 26 is large enough to prevent leakage from the garment 10. The design of top sheet 24 and back sheet 26 is known in the art, and one of ordinary skill in the art will be able to produce an appropriate top sheet 24 and an appropriate back sheet 26 without undue experimentation.

The top sheet 24 and the back sheet 26 may be associated with one another using a variety of methods known in the art. For example, they may be thermally, ultrasonically, or chemically bonded to one another. They also may be joined using a hot melt adhesive or mechanical fasteners, such as thread, clips, or staples. In one embodiment, a hydrophilic adhesive, such as CYCLOFLEX, sold by National Starch and Chemical Company, a corporation headquartered in Bridgewater, N.J., is used to join the top sheet 24 to the back sheet 26. The particular joining method may be dictated by the types of materials selected for the top sheet 24 and back sheet 26.

As mentioned above, absorbent garment preferably is provided with leg elastics 30 extending through crotch region 16, adjacent crotch edge 22. The absorbent garment of the invention also preferably is provided with waist elastics material 32 optionally in the first and second waist regions, 12, 14, respectively, to enable and assist in stretching around the wearer. The waist elastic materials 32 may be similar structures or different to impart similar or different elastic characteristics to the first and second waist regions 12, 14 of the garment. In general, the waist elastic materials may preferably comprise foam strips positioned at the first and second waist regions 12, 14, respectively. Such foam strips preferably are about ½ to about 1½ inches wide and about 3-6 inches long. The foam strips preferably are positioned between the top sheet 24 (or panels 301, 302, 303) and the back sheet 26. Alternatively, a plurality of elastic strands may be employed as waist elastics rather than foam strips. The foam strips preferably are comprised of polyurethane, but can be any other suitable material that decreases waist band roll over, reduces leakage over the waist ends of the absorbent garment, and generally improve comfort and fit. The first and optional second waist foam strips preferably are stretched 50-150%, preferably 100% more than their unstretched dimension before being adhesively secured between the back sheet 26 and top sheet 24. p Each edge 22 that forms the leg openings preferably is provided with adjacent leg elastics 30 to form a containment system. In the preferred embodiment, three strands of elastic threads (only two strands are shown in FIG. 2 for purposes of clarity) are positioned to extend adjacent to leg openings between the outer top sheet portions or panels 302, 303, and the back sheet 26. Any suitable elastomeric material exhibiting at least an elongation (defined herein as (LS-LR)/LR where LS is the stretch length of an elastic element and LR is retracted length, multiplied by 100 to obtain percent elongation) in the range of 5%-350%, preferably in the range of 200%-300%, can be employed for the leg elastics 30. The leg elastics 30 may be attached to the absorbent article 10 in any of several ways which are known in the art. For example, the leg elastics 30 may be ultrasonically bonded, heat/pressure sealed using a variety of bonding patterns, or glued to the garment 10. Various commercially available materials can be used for the leg elastics 30, such as natural rubber, butyl rubber or other synthetic rubber, urethane, elastomeric materials such as LYCRA (INVISTA, Inc., Wilmington, Del.), S-72 (Radici Spandex, Fall River, Mass.) or SYSTEM 7000 (Fulflex, Inc., Lincoln, R.I.).

The fastening elements, preferably a fastening system 34 (e.g., tab 34) of the preferred embodiment, is attached to the first waist region 12, and it preferably comprises a tape tab or mechanical fasteners 36. However, any fastening mechanism known in the art will be acceptable. Moreover, the fastening system 34 may include a reinforcement patch below the front waist portion so that the diaper may be checked for soiling without compromising the ability to reuse the fastener. Alternatively, other absorbent article fastening systems are also possible, including safety pins, buttons, and snaps.

As stated previously, the invention has been described in connection with a diaper. The invention, however, is not intended to be limited to application only in diapers. Specifically, the absorbent cores of the preferred embodiments may be readily. adapted for use in other absorbent garments besides diapers, including, but not limited to, training pants, feminine hygiene products and adult incontinence products.

The underlying structure beneath the top sheet 24 may include, depending on the diaper construction, various combinations of elements, but in each embodiment, it is contemplated that the absorbent garment will preferably include a absorbent core 28 comprising multiple layers between the top sheet 24 and back sheet 26. In addition, one or more additional layers 29 may be disposed between the top sheet 24 and absorbent core 28, and/or other additional layers may be disposed between these layers, or between absorbent core 28 and back sheet 26. An additional layer 29 also may be included in the absorbent core 28. The additional layer(s) 29 may include a fluid transfer layer, a fluid handling layer, a storage layer, a wicking layer, a fluid distribution layer, and any other layer(s) known to those having ordinary skill in the art.

Although the absorbent core 28 depicted in FIG. 1 has a substantially rectangular cross-sectional and plan view shape, other shapes may be used, such as a “T” shape or an hourglass shape. The shape of the absorbent core 28 may be selected to provide the greatest absorbency with a reduced amount of material. The absorbent core may be associated with the top sheet 24, back sheet 26, or any other suitable part of the garment 10 by any method known in the art, in order to fix the absorbent core 28 in place. In addition to the respective layers in the absorbent core 28, the overall absorbent core 28 may be enclosed within a tissue wrapping, as disclosed in U.S. Pat. No. 6,068,620 , the disclosure of which is incorporated by reference herein in its entirety. Persons of ordinary skill in the art are capable of designing and wrapping a suitable absorbent core 28 of the invention, using the guidelines provided herein.

The absorbent core 28 may extend into either or both of the first and second waist regions 12, 14. The absorbent core 28 of one preferred embodiment of the invention preferably includes at least three (3) layers whereby one of the layers is a central fibrous layer 284, another layer is a. three-dimensional extrudate layer, and a third layer is a base layer. The layers combined form a plurality of discrete containment wells 288 capable of retaining superabsorbent polymer.

In a preferred embodiment, the central fibrous layer 284 of absorbent core 28 comprises a fibrous structure. Central fibrous layers 284 of this type generally are known in the art, and exemplary absorbent cores are described in U.S. Pat. No. 6,068,620 and U.S. Pat. No. 5,281,207, both issued to Chmielewski, and U.S. Pat. No. 5,863,288, issued to Baker, the disclosures of each of which are herein incorporated by reference in their entirety and in a manner consistent with this disclosure.

Certain fibrous and particulate additives preferably are used as constituent elements of the central fibrous layer 284. Fibrous additives of central fibrous layer 284 preferably include, but are not limited to, synthetic fibers, such as cellulose ester fibers, cellulose acetate fibers, rayon fibers, lyocell fibers, polyacrylonitrile fibers, polyolefin fibers, surface-modified (hydrophilic) polyester fibers, surface-modified polyolefin/polyester bicomponent fibers, surface-modified polyester/polyester bicomponent fibers, or natural fibers, such as cotton or cotton linters, or combinations or blends thereof. The fibrous additives are preferably synthetic fibers. Of the foregoing, cellulose acetate is the most preferred synthetic fibrous additive for use in central fibrous layer 284. In addition, rayon, lyocell, and polyacrylonitrile have similar properties to cellulose acetate and are alternatively preferred. The remaining synthetic fibers, polyolefin fibers, surface-modified polyolefin/polyester bicomponent fibers, and surface-modified polyester/polyester bicomponent fibers are also believed to be effective fibrous additives.

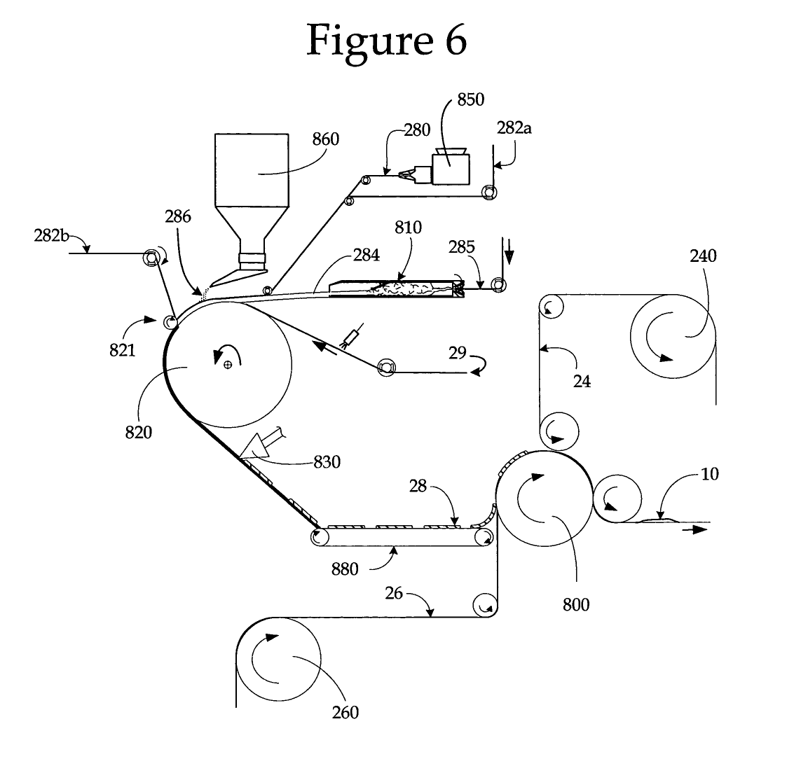

The synthetic fibrous component of the central layer 284 of absorbent core 28 preferably is comprised of tow fiber, and most preferably is a crimped tow of cellulose acetate, polypropylene, polyester, or mixtures thereof. Before making the absorbent core that includes a tow fiber, the tow fiber typically is unwound and opened, and then fed to the core forming station to provide a fibrous mass of material (see, FIG. 6). Persons of ordinary skill in the art are aware of techniques available to open tow fibers and form the opened fibers into a fibrous mass. In addition, the fibrous component of the central fibrous layer 284 may include a low-density roll good made in a separate process. Still further yet, the fibrous component could also include a carded web formed on-line. Optionally, it is advantageous to introduce from about 1-5% of a thermally bondable fiber into the fibrous component of the central fibrous layer 284 for wet strength and core stability in use. In addition to the tow material used as the fibrous component in central fibrous layer 284, other fibrous components also may be used.

In accordance with the present invention, the absorbent core preferably comprises a tow fiber, and preferably, a substantially continuous crimped filament tow. This fiber structure has high structural integrity, and as such, is distinct from a matrix of discontinuous fibers described as fluff, or fluff pulp in the prior art. The high structural integrity enables the production of stronger webs than those formed from discontinuous fibers, which in turn are believed to enable the production of thinner absorbent pads. In addition, the use of such fibers enables the production of ultra low density absorbent cores, when compared to absorbent cores prepared by dispersing SAP particles in fluff.

The synthetic fiber can be any substantially continuous or discontinuous thermoplastic filament fiber that is capable of being used in combination with SAP in an absorbent core. Preferably, polypropylene or cellulose ester fiber is used as the fibrous material in central fibrous layer 284. Non-limiting examples of suitable cellulose esters include cellulose acetate, cellulose propionate, cellulose butyrate, cellulose caproate, cellulose caprylate, cellulose stearate, highly acetylated derivatives thereof such as cellulose diacetate, cellulose triacetate and cellulose tricaproate, and mixtures thereof such as cellulose acetate butyrate. A suitable cellulose ester will include some ability to absorb moisture, (but absorptive capacity is not necessarily required), preferably is biodegradable, and is influenced not only by the substituent groups but also by the degree of substitution. The relationship between substituent groups, degree of substitution and biodegradability is discussed in W. G. Glasser et al., BIOTECHNOLOGY PROGRESS, vol. 10, pp. 214-219 (1994), the disclosure of which is incorporated herein by reference in its entirety.

The synthetic fiber useful in the present invention is beneficially moisture-absorbent and biodegradable. Accordingly, cellulose acetate tow is typically preferred for use in the invention. Typically, the denier per fiber (dpf) of the fiber will be in the range of about 1 to 25, preferably about 3 to 15, and most preferably about 6 to 7. For the same weight product, filaments of lower dpf may provide increased surface area and increased moisture absorption. Total denier may vary within the range of about 5,000 to 60,000, more preferably from about 20,000 to about 40,000, and most preferably from about 20,000 to about 30,000, depending upon the process used.

It is particularly preferred in the invention to use tow fiber having crimped filaments. Tow materials having crimped filaments are typically easier to open. Separation of filaments resulting from bloom advantageously results in increased available filament surface area for superabsorbent material immobilization and increased moisture absorption. Gel blocking also may be reduced by using crimped tow in the central fibrous layer 284. As therefore may be understood, more crimp is typically better, with in excess of about 20 crimps per inch being usually preferred. Substantially continuous filament; cellulose ester tow having crimped filaments with about 25 to 40 crimps per inch, is commercially available from Celanese Acetate, Charlotte, N.C.

It is preferred in the present invention that the tow fibers in central fibrous layer 284 have an average length generally about the same length as the absorbent core. Typically, the tow is a substantially continuous filament that is cut to length during manufacture of the core. The average diameter of the tow fibers typically is expressed as the cross sectional area of the fibers, although the width of the fibers preferably is within the range of from about 50 to about 200 mm, more preferably from about 75 to about 150 mm, and most preferably from about 85 to about 120 mm. The cross sectional area is based on the denier and density of the fibers. For example, the denier per foot (dpf) and density (typically an acetate polymer density is about 1.32 g/cm³), can be used to calculate the cross sectional area. A 3.0 dpf acetate polymer fiber has a cross sectional area 2.525×10⁻⁶ cm².

The central fibrous layer 284 may optionally comprise discontinuous synthetic fibers. As used herein, the term “discontinuous” fibers means fibers that have an average length less than the length of the absorbent core. As such, the central fibrous layer 284 may comprise, for example, a nonwoven mat or web of discontinuous synthetic fibers. The fibers may be provided to the absorbent core 28 as a substantially continuous tow fiber, and then cut to length and formed into a web during the processing of the absorbent core 28, or the fibrous web may be formed off-line, and provided to the absorbent core 28 as a roll-good material.

The central fibrous layer 284 may optionally contain superabsorbent polymer (SAP). Any superabsorbent polymer now known or later discovered may be used in central fibrous layer 284 so long as it is capable of absorbing liquids. Useful SAP materials are those that generally are water-insoluble but water-swellable polymeric substance capable of absorbing water in an amount that is at least ten times the weight of the substance in its dry form. In one type of SAP, the particles or fibers may be described chemically as having a back bone of natural or synthetic polymers with hydrophilic groups or polymers containing hydrophilic groups being chemically bonded to the back bone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated or phosphorylated, causing the SAP to be highly hydrophilic. Such modified polymers may also be cross-linked to reduce their water-solubility.

Examples of suitable SAP are water swellable polymers of water soluble acrylic or vinyl monomers crosslinked with a polyfunctional reactant. Also included are starch modified polyacrylic acids and hydrolyzed polyacrylonitrile and their alkali metal salts. A more detailed recitation of superabsorbent polymers is found in U.S. Pat. No. 4,990,541 to Nielsen, the disclosure of which is incorporated herein by reference in its entirety.

Commercially available SAPs include a starch modified superabsorbent polymer available under the trade name HYSORB® from BASF Aktiengesellschaft, Ludwigshafen, Germany. Other commercially available SAPs include a superabsorbent derived from polypropenoic acid, available under the tradename DRYTECH® 520 SUPERABSORBENT POLYMER from The Dow Chemical Company, Midland Mich.; AQUA KEEP, and AQUA KEEP SA60S, manufactured by Sumitomo Seika Chemicals Co., Ltd., Osaka Japan.; ARASORB manufactured by Arakawa Chemical (U.S.A.) Inc.; FAVOR manufactured by Stockhausen Inc.; DIAWET, commercially available from Mitsubishi Chemicals, Japan; FLOSORB, available from SNF Floerger, France, AQUALIC, available from Nippon Shokubai, Osaka, Japan.

The SAP may be provided in any particle size, and suitable particle sizes vary greatly depending on the ultimate properties desired. It has been known to prepare absorbent cores comprised of cellulose acetate tow or other polymeric fibers and SAP, as described in H1565, and U.S. Pat. Nos. 5,436,066, and 5,350,370, the disclosures of each of which are incorporated by reference herein in its entirety.

It is preferred in a SAP-containing central fibrous layer 284 to use relatively coarse fibers having a low basis weight such that the pore size of the matrix formed by the mass of tow fibers does not entrain some or most of the SAP, but rather allows the SAP to fall freely through the matrix. The basis weight of preferred fibers used in the present invention ranges from about 20 to about 200 g/m², more preferably from about 50 to about 100 g/m², and most preferably from about 70 to about 80 g/m².

The concentration of fibrous material in the central layer 284 of the absorbent core 28 of the invention preferably is about 5%-99%, more preferably about 80%-99%, and most preferably about 90%-99%. Most preferably, the central fibrous layer 284 comprises from about 0%-50% SAP and from about 50%-99% fibrous materials selected from the foregoing group, or the fibrous components discussed below.

Particulate additives may be added to central fibrous layer 284 in addition to or as a substitute for the foregoing fibrous additives in order to maintain high SAP efficiency. The particulate additives preferably are insoluble, hydrophilic polymers with particle diameters of 100 μm or less. The particulate additives are chosen to impart optimal separation of the SAP particles. Examples of preferred particulate additive materials include, but are not limited to, potato, corn, wheat, and rice starches. Partially cooked or chemically modified (i.e., modifying hydrophobicity/hydrophilicity, softness, and hardness) starches can also be effective. Most preferably, the particulate additives comprise partially cooked corn or wheat starch because in this state, the corn or wheat are rendered larger than uncooked starch and even in the cooked state remain harder than even swollen SAP. In any event, regardless of the particulate additive chosen, one of the many important criteria is to use particulate additives that are hard hydrophilic materials relative to swollen SAP or which are organic or inorganic polymeric materials about 100 microns in diameter. Fibrous and particulate additives can be used together in these absorbent laminates. Examples of SAP/particulate and SAP/fiber/particulate additives include those described in, for example, U.S. Pat. No. 6,068,620.

If desired, an absorptive pad of multiple layer thickness, may be provided. To this end, the tow may be, for example, lapped or crosslapped in accordance with conventional procedures. In this way, a superabsorbent, absorptive material of a desired weight and/or thickness may be provided. The specific weight or thickness will depend upon factors including the particular end use. It is especially preferred that the crimped cellulose acetate tow material be opened and then mixed with the SAP particles to form the central fibrous layer 284.

Optionally, about 1-10%, preferably about 5%, by weight of thermally bondable synthetic fibers can be added to the absorbent core 28 to impart additional wet strength to the laminate. This will improve the stability of the core during use of the diaper. The preferred synthetic fibers are polyolefin/polyester fibers and polyester/polyester bicomponent fibers.

Disposed beneath the central fibrous layer 284 of the absorbent core 28 is a three-dimensional extrudate layer 280, and one or more base layers 282. The three-dimensional extrudate layer 280 provides a three-dimensional structure having voids or depressions therein, which form a portion of the plurality of discrete containment wells 288 that contain superabsorbent particles in the absorbent core. The three-dimensional extrudate layer 280 may be formed from any extrudable material capable of being melted and extruded under pressure through a die or orifice to form a continuous shape such as a thin film, filament, fiber, or fragments or combinations thereof. Examples of extrudable materials include, for example, waxes, thermoplastics, hotmelt adhesives and the like. The extrudate layer 280 may be formed by any method capable of providing the extrudate in a form that has a three-dimensional structure capable of creating a plurality of discrete containment wells 288 as described herein. The extrudate, whether filament or film, forms the side walls or perimeter of discrete containment wells 288, while the void areas (open areas) and the thickness of extrudate layer determine the volume of each containment well 288. For instance, where the extrudate layer 280 is formed from one or more extruded filaments, the perimeter of the containment wells 288 is defined by the filaments, and the depth of the containment wells 288 is defined by the thickness of the filament. Therefore, it is preferable that the three-dimensional extrudate layer 280 has an open area sufficient to contain a predetermined amount of SAP to provide the desired absorbency in the absorbent core 28. For instance, in some preferred embodiments, the extrudate layer 280 has an open area of about 50% to about 99%. It is also preferable that the three-dimensional extrudate layer 280 has a height sufficient to contain multiple SAP particles. For instance, in some preferred embodiments, the extrudate layer 280 has a height of about 100 μ to about 3000 μ.

There are several extrusion processes available that are capable of providing an extrudate layer 280 as described. For example, the extrudate layer 380 may be provided using a hot melt spiral spray process that produces a substantially continuous spiral pattern forming discrete open areas; or a random fiberization spray that produces a plurality of extruded fibers that intersect to form discrete void areas. Alternately, the extrudate layer 380 may be printed onto a substrate, such as with a screen-printing or gravure-style printing process, providing a substantially continuous extrudate layer having void areas therein. One of ordinary skill in the art would be able to design a process capable of delivering the extrudate layer 280 in accordance with the direction given herein.

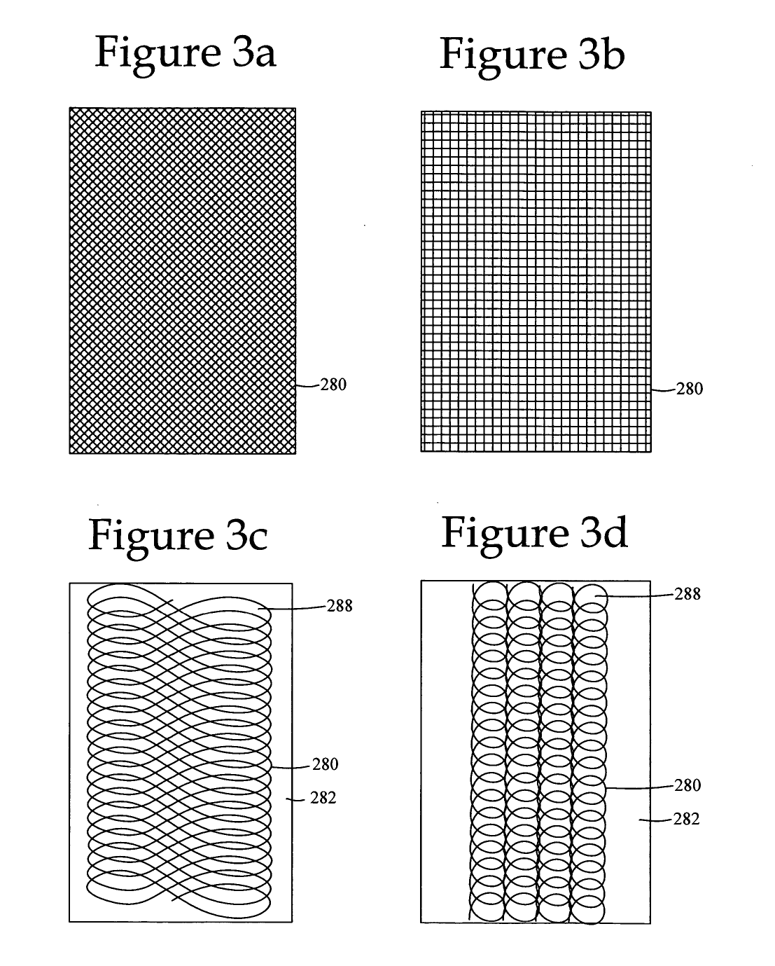

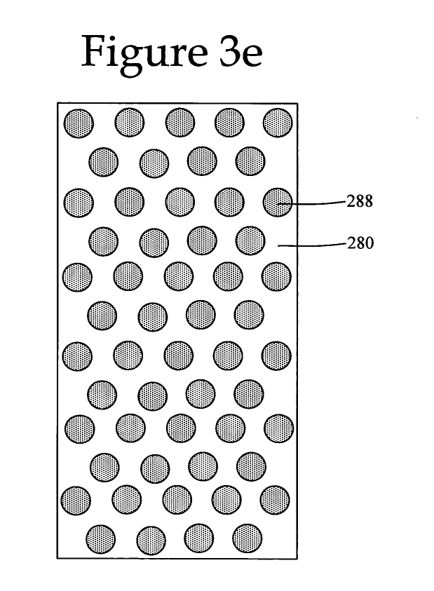

The three-dimensional extrudate layer 280 may be provided in any pattern capable of providing sufficient structure and open area for containing SAP particles. Obviously, there are a multitude of pattern configurations that are capable of providing an extrudate layer 280 with the described features. By way of example, FIGS. 3a to 3e show some possible configurations. FIGS. 3a and 3b show a cross-hatch pattern, whereby the extrudate is formed by a network of parallel, intersecting filaments of extrudate. The extrudate filaments form the side walls of a containment wells 288, while the void areas between the filaments provide areas for the containment of superabsorbent material. Alternately, the extrudate may be provided with one or more continuous spiral spray patterns with open areas that form containment wells 288, such as those shown in FIGS. 3c and 3d. As shown in FIG. 3c, the extrudate layer 280 may include only a single substantially continuous filament, or alternately, as. shown by FIG. 3d, may include multiple filaments of adhesive. In another embodiment of the invention, as shown in FIG. 3e, the extrudate may be provided in a substantially. continuous layer with void areas provided in the layer to provide discrete containment wells 288 for containing SAP. One of ordinary skill in the art should be capable of designing an extrudate pattern which provides the desired features of the invention using the guidance provided herein.

One or more base layers 282 also is included in the absorbent core 28. The purpose of the base layer 282 is to provide a substantially continuous substrate to the base surface of the discrete containment wells 288. Preferably, the extrudate layer 280 is in intimate contact with the base layer 282—when combined this way, the extrudate layer 280 provides the perimeter and depth of the containment wells 288, while the base layer 282 provides the base of the containment wells 288. In certain embodiments, the extrudate layer 280 is provided to the absorbent core 28 on a base layer 282. For example, the extrudate layer 280 may be extruded or printed directly to the base layer 282 prior to introduction to the absorbent core 28. Alternately, the base layer 282 and the extrudate layer 280 may be separately provided. The extrudate layer 280 may be attached to the base layer 282 using any technique known in the art or later discovered including, for example, adhesive bonding, thermal bonding, compression bonding, or the like. It is important to the invention, however, that the base layer 282 and the extrudate layer 280 are in intimate contact, in order to provide a discrete enclosure for containing the SAP particles.

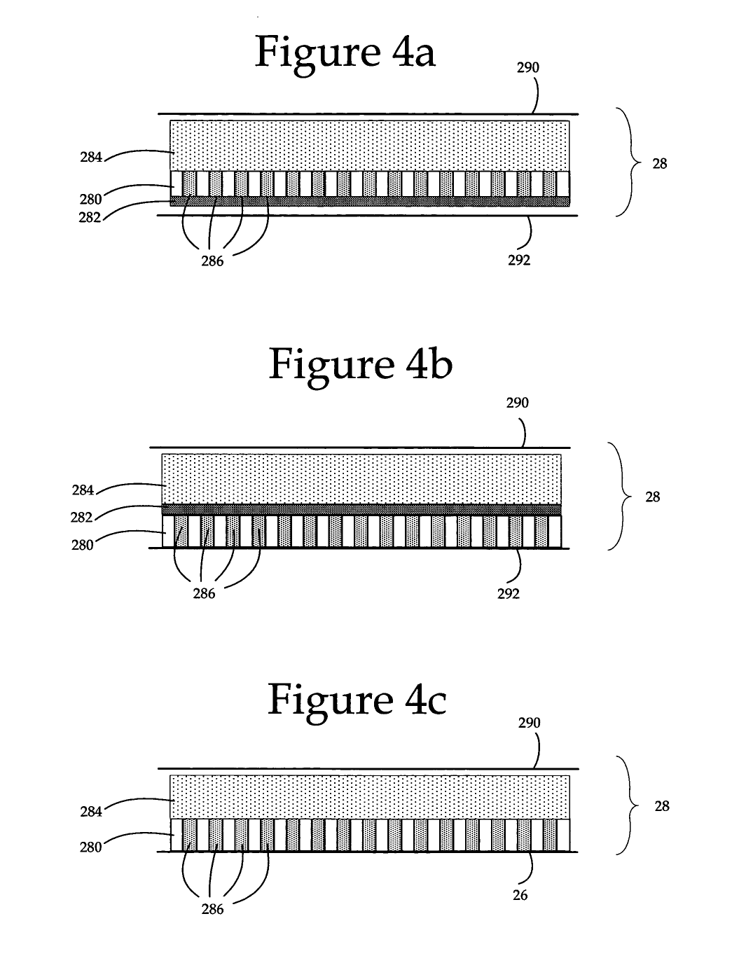

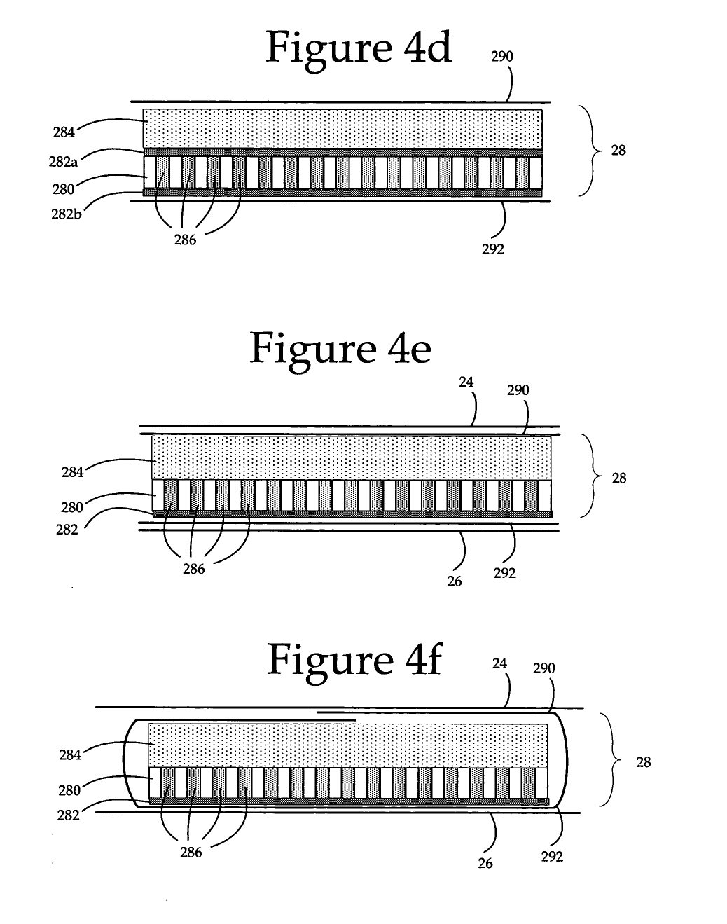

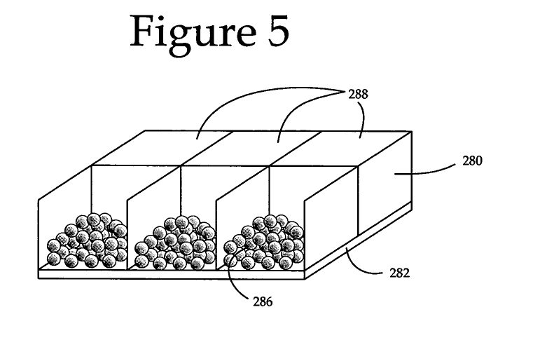

The base layer 282 may be comprised of any material capable of forming a base surface for the containment wells 288, and containing SAP particles 286. For example, the base layer material may comprise a tissue, a nonwoven, a film, a substantially continuous layer of extrudate, or combinations or fragments thereof. The base layer 282 may be disposed below the extrudate layer (as shown in FIG. 4a) or on top of the extrudate layer 280 (as shown in FIG. 4b). In some embodiments, the base layer material 282 may comprise a portion of the back sheet 28 (as shown in FIG. 4c) or other layer, such as additional layers 29, provided in the absorbent article 10. In other embodiments, two base layers may be provided (282a, 282b in FIGS. 4d and 4e), where at least one of the base layers 282a, 282b being in intimate contact with the extrudate layer 280. For example, as shown in FIG. 4d, the two base layers 282a, 282b are disposed directly on top of and beneath the extrudate layer. In an alternate embodiment, as shown in FIG. 4e, base layer 282b is provided in direct contact with the extrudate layer 280, while the other base layer 282a is disposed on top of the central fibrous layer 284. In yet another embodiment, a single base layer 282 is provided, where this base layer wraps around the absorbent core 28, enclosing both the extrudate layer 280 and the central fibrous layer 284 (as shown in FIG. 4f).

When the extrudate layer 280 and the base layer 282 are combined, they form three-dimensional containment wells 288 where the extrudate layer 280 forms the side walls of the containment wells 288, and the base layer forms the base of the containment wells 288. When SAP particles 286 are provided to the absorbent core 28, they may be dispersed in these containment wells 288. For example, FIG. 5 shows a cutaway of a network of containment wells 288 formed by base layer 282 and extrudate layer 280, containing SAP particles 286. The discrete containment cells contain and segregate the SAP particles 286, preventing them from migrating to other portions of the absorbent article.

In certain embodiments of the invention, after the SAP particles 286 have been deposited in the containment wells 288, the extrudate layer 280 is attached (on the surface opposite the base layer 282) to another layer, thereby “closing” the containment wells 288. The extrudate may be attached to this layer or substrate using any technique known or later discovered in the art including, for example, adhesive bonding, thermal bonding, compression bonding, or the like. In a preferred embodiment, the extrudate layer 280 is disposed directly beneath central fibrous layer 284, and directly above base layer 282 (as shown in FIG. 4a). In this embodiment, each of the discrete containment wells 288 are defined on the top by the central fibrous layer 284, on the sides by the extrudate layer 280, and on the base by the base layer. SAP particles 286 are provided in the discrete containment wells 288, and are prevented from moving or shifting to other portions of the absorbent core 28. It is also possible for the extrudate layer 280 to be attached to other materials, such as a second base layer (282a shown in FIG. 4d), or an additional layer 29, or any other materials that are capable of enclosing the SAP particles within the containment wells, such as, for example, a tissue layer, a non-woven layer, a back sheet layer, a wicking layer, a fluid transfer layer, a fluid handling layer, a storage layer, a fluid distribution layer, or combinations and fragments of these layers. Regardless of the specific configuration of the layers that form the containment wells 288, it is preferable that the substrate bonded on the top surface of the extrudate layer 280 is liquid permeable, so that that fluid may penetrate to the SAP 286 contained in the containment wells 288.

The containment wells 288 are beneficial to the absorbent core 28, because they allow for precise deposition of SAP particles 286 within the absorbent core 28. The three-dimensional structure provides a plurality of discrete zones in which SAP may be deposited. This allows a designer to more precisely select the regions in which to place SAP particles 286 within the absorbent core 28. In addition, the consistency of placement of the SAP particles 286 may be improved because the containment wells 288 confine the SAP 286 to the regions in which they are deposited. Another benefit provided by the use of the containment wells 288 is that after initial placement, the SAP particles 286 are enclosed within the containment wells 288 and prevented from shifting to other parts of the absorbent core 28, especially during distribution of the product and normal use conditions. This, in turn, provides more consistent absorbency performance.

In certain embodiments, it is preferable that some or all of the containment wells 288 have a tacky inner surface, to which the SAP particles 286 adhere. This may be achieved by forming the extrudate layer 280 and/or the base layer 282 from a hot melt adhesive or other tacky extrudate. Alternately, an adhesive may be added to the containment wells 288 prior to introduction of the SAP particles 286. In another embodiment, the extrudate layer 280 comprises a material that swells upon contact with liquid. In this embodiment, the containment wells 288 may grow in height to allow for swelling of the SAP 286 as it absorbs the liquid.

The foregoing absorbent cores 28 of the preferred embodiments preferably are made using a dry process, whereby the respective components of the composite core 28 are brought together in a dry state, as opposed to one or more components being in a liquid state. Persons of ordinary skill in the art will be capable of making the absorbent cores 28 of the present invention, using the guidelines provided herein.

The total basis weight of the absorbent core 28 including fibrous materials, SAP, extrudate layer, base layers, additional layers, and additives, can be anywhere from about 50-1,000 grams per square meter. The most preferred total basis weights of the absorbent core 28 are about 300-600 grams per square meter.

In addition to the other configurations, additional layers may be present in the absorbent core 28. For example, absorbent core 28 may include an additional layer 29 disposed above, below or between any of layers of the absorbent core 28, such as above the central fibrous layer 284, and/or below central fibrous layer 284. Any additional layer 29 can be used, including any layer selected from a fluid acquisition layer, a distribution layer, an additional fibrous layer optionally containing SAP, a wicking layer, a storage layer, or combinations and fragments of these layers. Such layers may be provided to assist with transferring fluids to the absorbent core 28, handling fluid surges, preventing rewet, containing absorbent material, improving core stability, or for other purposes. Persons of ordinary skill in the art are familiar with the various additional layers 29 that may be included in an absorbent article, and the present invention is not intended to be limited to any particular types of materials used for those layers. Rather, the invention encompasses all types of wicking layers, all types of distribution layers, etc., to the extent that type of layer is utilized. Furthermore, any of those additional layers 29 described herein can be used as base layer 282 as long as it is capable of containing SAP particles 286 within the containment wells 288.

As shown in FIGS. 4a-4f, the absorbent core 28 may contain upper and lower layer 290, 292, which encase the central fibrous layer 284, extrudate layer 280, base layer 282, and SAP 286. These layers 290, 292 may be made of, for example, tissue, film or nonwoven, but may also form the top sheet and back sheet of the absorbent garment, or any other layers. The upper and lower layers 290, 292, preferably. are wider than the central fibrous layer 284 that forms the absorbent core, and their side portions preferably are sealed to one another by bonding, by crimping or by both to prevent release of opened tow and particles of SAP. As shown in FIG. 4f, the upper and lower layers 290, 292, preferably are comprised of the same material folded over onto itself, and only the open end sealed by crimping or bonding. The absorbent core 28, comprising the assembly of the central fibrous layer 284, extrudate layer 280, base layer 282, including the opened tow and SAP, may be further processed as it is conveyed through the assembly line for inclusion into absorbent garments. For example, the absorbent core 28 may be severed into individual absorbent cores, and the severed ends may be crimped or bonded or both to prevent the SAP from exiting the ends.

Crimping, bonding or both can be performed on the absorbent core 28 of the invention using conventional means. For example, the lateral side edges, and longitudinal edges can be sealed together by intermittent or substantially continuous application of adhesive to the respective portions of the upper and lower layers 290, 292 using any device capable of applying adhesives to a continuous moving web of material. The lateral and/or longitudinal edges then can be pressed together to form a seal. The seal also can be formed ultrasonically, or the respective edges (lateral and/or longitudinal) can be crimped using crimping rollers or any other crimping device known to those having ordinary skill in the art. Using the guidelines provided herein, those skilled in the art will be capable of sealing the lateral and/or longitudinal edges of absorbent core 28 using bonding, crimping, or both.

It is possible in the present invention to mechanically and/or thermally work the absorbent core 28 to make it more flexible. Any technique presently known in the art or later discovered may be used to work the absorbent core. For instance, the absorbent core 28 may be embossed or texturized using a continuous or intermittent calendaring apparatus. Other useful techniques include, for example, compression, thermal bonding, and ultrasonic bonding. Optionally, the top sheet 24 and/or the back sheet 26 may be worked with the absorbent core 28.

It is also possible in the present invention that the absorbent core 28 be folded as it is disposed in the absorbent garment. The absorbent core 28 can be folded in any suitable manner, including any and all of those disclosed in U.S. Pat. No. 6,068,620. Suitable folds include “C” folds, “G” folds, “U” folds, “A” folds, pleats or “W” folds, and the like.

The invention also relates to a method of making an absorbent core 28, and an absorbent article 10 that includes providing a top sheet material 24 and a back sheet material 26. The method also includes preparing an absorbent core 28 that contains a three-dimensional extrudate layer 280, a base layer 282, and a central fibrous layer 284 comprised of tow fiber. When combined, the three layers form containment wells 288 that contain SAP particles 286 within the absorbent core 28. The method includes disposing the absorbent core 28 between the top sheet 24 and the back sheet 26. The method provides an absorbent core 28 having precise placement of SAP particles 286, throughout the cross-section of the absorbent core 28. This allows for either select regions of increased absorbency due to the presence of varying concentrations of SAP, or improved uniformity of absorbency, when the SAP concentrations are kept constant throughout the cross-section of the absorbent core.

Preparing the absorbent core includes forming the three-dimensional extrudate layer 280, and combining it with a base layer 282 so that the two layers are in intimate contact to form a plurality of containment wells 288. The method further includes depositing SAP particles 286 within the containment wells 288, and then laminating another layer on the open side of the three-dimensional extrudate layer 280, to fully enclose the deposited SAP particles 286 within the containment wells 288. The layer laminated to the open side of the three-dimensional extrudate layer 280 may be a central fibrous layer, a base layer, or another layer, such as an additional layer. The method optionally includes enclosing the absorbent core between upper and lower layers 290, 292, such as tissue layers, to enclose and contain the absorbent materials. In one embodiment of the invention, the SAP particles 286 are distributed so that the target absorbency zone has a higher concentration of SAP than other areas of the absorbent core.

FIGS. 6, 7 and 8 illustrate apparatuses useful in forming an absorbent article 10 in accordance with the present invention. Any type of synthetic fiber 285, or mixtures of synthetic fibers 285, can be supplied to the apparatus. Preferably, the synthetic fiber 285 is provided as a substantially continuous tow fiber and, as conventional in the art, the synthetic tow fiber 285 typically is opened to form central fibrous layer 284. In this regard, the apparatus includes a tow opener and feeder 810 that is capable of opening any suitable tow material, expanding the tow fiber and feeding the tow fiber to the core forming station 820. Any suitable tow opener and feeder 810 can be used in the method of the invention. Preferably, the tow opener and feeder 810 is capable of opening a plurality of different tow fibers (e.g., varying denier, coarseness, chemical make-up, etc.) and feeding the fibers to the core forming station 820. For example, the apparatus may include two or more tow opener devices 810, that feed the tow to a common nozzle (not shown) that distributes the combined synthetic tow fibers 285 to the core forming station 820. Optionally, the synthetic fibers 285 may be provided as a pre-formed roll-good material, thereby obviating the need for the tow opener and feeder 810.

The synthetic fibers 285 optionally are mixed with superabsorbent polymer (SAP) material 286. The SAP 286 may be fed to and mixed with the synthetic fibers 285 by known or later-developed method. Persons of ordinary skill in the art are capable of designing a suitable SAP feeder and nozzle configuration to provide adequate mixing of SAP material 286 and synthetic fibers 285 to form central fibrous layer 284.

The extrudate layer 280 is provided by any extruder apparatus 850 capable of producing a three-dimensional extrudate layer having a plurality of voids therein. Extruders typically include a melting mechanism for melting the extrudate, and a feed mechanism for transporting the molten extrudate under pressure to a die or orifice to form a substantially continuous shape such as a thin film, filament, or fiber. For instance, the extruder apparatus 850 may comprise of a melter, a pump and spray nozzle, whereby the apparatus 850 extrudes a plurality of fibers to form a network of intersecting fibers, defining a plurality of void spaces. As another example, the extruder apparatus 850 may comprise a melter, a pump and an orifice which feeds to a gravure printer, whereby the printer produces a pattern of extrudate defining a plurality of void spaces. Alternate mechanisms for producing a three-dimensional extrudate layer are contemplated, including, for example, thin film extruders, bead extruders and screen printers. One of ordinary skill in the art is capable of designing an apparatus to provide a three-dimensional extrudate layer according to the guidance provided herein.

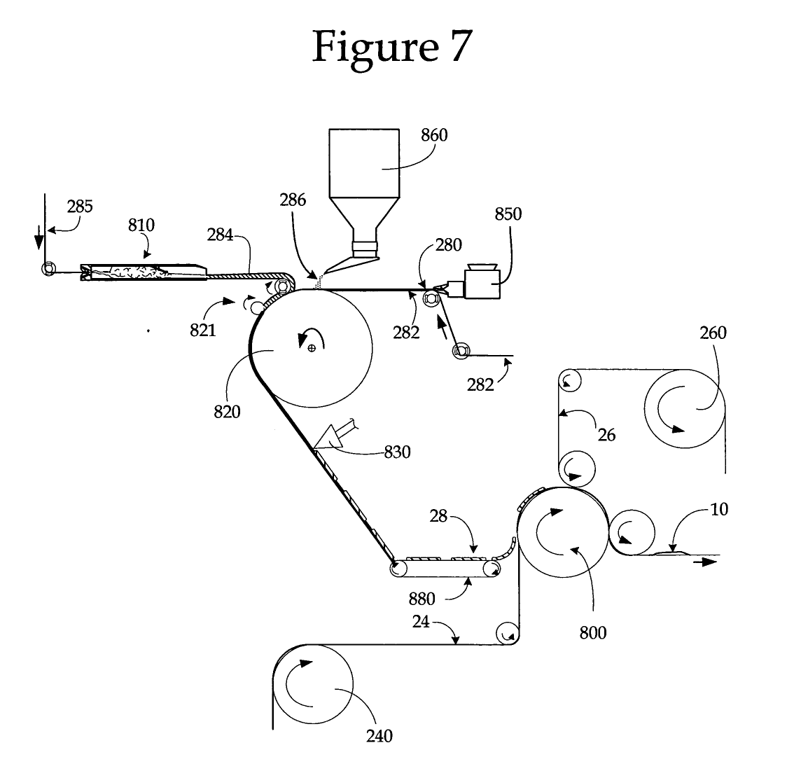

As shown in FIG. 7, the extrudate layer 280 may be formed directly onto a base layer 282, while the extrudate is in a molten, or semi-molten state. For example, the extrudate may be sprayed onto the base layer 282, or it may be screen printed onto the base layer 282, while the extrudate is still substantially in molten state. The base layer 282 therefore acts as a carrier layer to supply the extrudate layer 280 to the core forming station 820. Forming the extrudate layer 280 directly to the base layer 282 also helps to provide intimate contact between the extrudate and the base layer 282. Alternately, the extrudate layer 280 may be provided as a separate layer to be later combined with base layer 282. The process may also include an apparatus for cooling or setting the extrudate layer 280 to a solid or semi-solid state.

Preferably, base layer 282 is provided as a separate roll good material, and is transported to the extruder apparatus 850 or the forming station 820 by a supply mechanism, which can be any supply mechanism known in the art. Preferably, the base layer 282 material is supplied via a supply roller and select feed an/or guide rollers (not shown). Alternately, the base layer 282, may be provided by an extrusion process, providing a substantially continuous thin film or network of fibers, or the like, as described above in relation to the extrudate layer 280. When the base layer 282 is extruded, it is preferable that the extruded material is sufficiently continuous to contain SAP particles within the containment wells 288. In certain embodiments, a second base layer may be provided to the absorbent core (for example, see FIG. 6), where the second base layer is similarly provided.

It is important that the base layer 282 and the extrudate layer 280 are in intimate contact to form containment wells 288. Where the extrudate layer 280 and the base layer 282 are provided as separate layers, the two layers may be bonded or laminated using any technique known in the art, or later-discovered. For instance, adhesive can be applied to either the extrudate layer or the base layer, or to both, by an adhesive applicator. Again, any mechanism capable of supplying an adhesive, albeit a spray adhesive, or one that is slot-coated on, can be used in the invention. Suitable adhesives include any adhesive commonly employed in absorbent garments that is useful in adhering one or more tissue and/or non-woven materials together. It is particularly preferred to use construction adhesives, including, for example, HL-1258 by H. B. Fuller Company of St. Paul, Minn.; H2587-01 by AtoFindley, Inc., of Wauwatosa, Wis.; and NS 34-5665 by National Starch and Chemical Co. of Bridgewater, N.J. Any of these adhesives may be used in all adhesive applications in the absorbent garment, or only in select applications as a construction adhesive for bonding parts of the garment as the top sheet, back sheet, absorbent core, and additional layers.

The positioning and amount of adhesive can be altered either during line down time, or during manufacture of absorbent article 10, by controlling the positioning of adhesive applicator with an adhesive applicator controller. Any system can be used to control the amount, if any, and location of application of the adhesive. Those skilled in the art are capable of designing a suitable adhesive applicator to apply select amounts of adhesive to the desired portions of the extrudate layer 280 or base layer 282, using the guidelines provided herein.

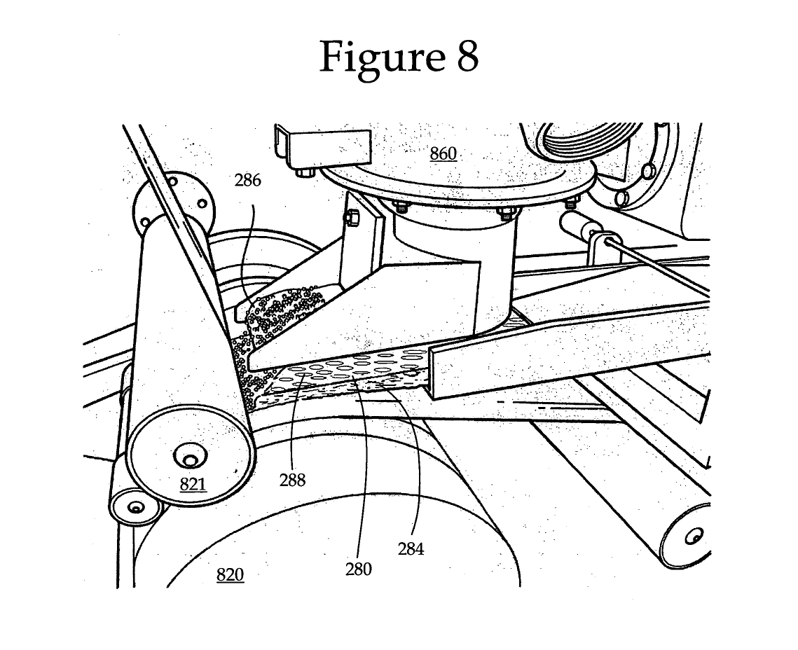

The laminate of the base layer 282 to the extrudate layer 280 forms a continuous substrate having plurality of open containment wells 288. This laminate is fed to the core forming station, where SAP is provided from SAP feeder 860, as shown in FIG. 8. The SAP is fed to the core forming station 820 by any SAP feeder 860 capable of feeding the SAP to the core forming station 820. Persons of ordinary skill in the art are capable of designing a suitable SAP feeder 860 and nozzle configuration to provide adequate distribution of SAP material 286 to the containment wells 288. The SAP particles may be distributed evenly amongst the individual containment wells 288, or may be distributed so that SAP is localized in one or more selected regions of the absorbent core 28.

Once the SAP has been deposited in the containment wells, the open (un-laminated) surface of the extrudate layer is bonded to another substrate to close containment wells 288. In doing so, the extrudate layer may be attached or laminated to a central fibrous layer 284 (as shown in FIG. 7), a second base layer (such as 282b in FIG. 6), the backsheet layer 26, lower layer 292, or another layer such as additional layer 29. The bonding may be provided by any method known in the art, such as adhesive bonding (described above), heat bonding or pressure bonding, or a combination of two or more of bonding methods. Once the extrudate is bonded to the substrate, the containment wells 288 encase and segregate substantially all of the SAP particles deposited therein so that they do not migrate or shift from their original position.

The multiple layers of absorbent core 28 are combined at forming station 820. In addition to the extrudate layer 280, base layer(s) 282, 282a, 282b, central fibrous layer 284, other layers such as upper and lower layers 290, 292 and additional layer(s) 29 may be provided to the absorbent core 28 at forming station 820. The multiple layers of absorbent core 28 may then become affixed when the absorbent core 28 is passed through the one or more nip rollers 821 at the core forming station 820.

The absorbent cores 28 then are cut to length by cutting knife 830. Cutting knife 830 can be any suitable cutting device capable of cutting absorbent core 28 of the invention. For example, cutting knife 830 can be comprised of a set of rollers; one being an anvil, and another having a knife attached at one point on the roller, whereby the diameter of the roller is selected to coordinate with the speed at which absorbent cores 28 are formed. The knife roller and anvil roller then can rotate at the same speed as the line speed to cut the absorbent core 28 at select areas to form uniform length cores 28. Optionally, the knife roller apparatus may be equipped to crimp or seal the ends of the absorbent cores 28 during the cutting process. Persons of ordinary skill in the art are capable of designing a suitable cutting knife 830 given the specifics of each article forming assembly line.

The absorbent cores 28 then are transported to forming station 800 via core conveyor 880. Top sheet material 24 may be supplied to forming station 800 by top sheet supply mechanism 240, which can be any supply mechanism capable of supplying top sheet 24 to forming station 800. Preferably, top sheet material 24 is supplied via a supply roller 240 and select feed and/or guide rollers (not shown). Back sheet material 26 likewise can be supplied to forming station 800 by back sheet supply mechanism 260, which can be any supply mechanism capable of supplying back sheet 26 to forming station 800. Preferably, back sheet material 26 is supplied via a supply roller 260 and select feed and/or guide rollers (not shown). Forming station brings together the respective components of absorbent article 10 by disposing absorbent core 28 between top sheet material 24, and back sheet material 26. The final absorbent article 10 then may be cut and folded to the appropriate size and shape downstream from forming station 800.

Other embodiments, uses, and advantages of the invention will be apparent to those skilled in the art from consideration of the specification and practice of the invention disclosed herein. The specification should be considered exemplary only, and the scope of the invention is accordingly intended to be limited only by the following claims.

1. An absorbent core for a disposable absorbent article comprising:
a central fibrous layer comprising synthetic fibers;
a three-dimensional extrudate layer having a plurality of voids;
a base layer; and
superabsorbent polymer;
wherein the three dimensional extrudate layer, and the base layer form a plurality of discrete containment wells in which at least some of the superabsorbent polymer is contained.

2. The absorbent core of claim 1, where the plurality of discrete containment wells further comprise at least a portion of the central fibrous layer.

3. The absorbent core of claim 1, where the plurality of discrete containment wells further comprise a second base layer.

4. The absorbent core of claim 1, where the plurality of discrete containment wells further comprise a layer selected from the group consisting of: a tissue layer, a non-woven layer, a back sheet layer, a wicking layer, a fluid transfer layer, a fluid handling layer, a storage layer, a fluid distribution layer, and combinations and fragments thereof.

5. The absorbent core of claim 1, where the synthetic fibers are selected from the group consisting of cellulose ester fibers, cellulose acetate fibers, rayon fibers, lyocell fibers, polyacrylonitrile fibers, polyester fibers, polypropylene fibers, polyethylene fibers, and mixtures and combinations thereof.

6. The absorbent core of claim 5, where the synthetic fibers are a cellulose ester fibers.

7. The absorbent core of claim 5, where the synthetic fibers are cellulose acetate fibers.

8. The absorbent core of claim 5, where the synthetic fibers are polypropylene fibers.

9. The absorbent core of claim 1, where the synthetic fibers are substantially continuous fibers.

10. The absorbent core of claim 1, where the synthetic fibers are tow fibers.

11. The absorbent core of claim 9, where the length of the synthetic fibers is substantially equal to the length of the absorbent core.

12. The absorbent core of claim 1, where the synthetic fibers are discontinuous fibers.

13. The absorbent core of claim 12, where the synthetic fibers are formed into a carded non-woven web.

14. The absorbent core of claim 1, wherein the absorbent core further comprises at least one additional layer.

15. The absorbent core of claim 14, where the at least one additional layer is selected from the group consisting of: a fluid transfer layer, a fluid handling layer, a storage layer, a wicking layer, a fluid distribution layer, and combinations and fragments thereof.

16. The absorbent core of claim 1, wherein the base layer comprises a materials selected from the group consisting of: an extrudate, a tissue layer, a nonwoven layer, a film layer, and combinations and fragments thereof.

17. The absorbent core of claim 16, wherein the base layer comprises an extrudate layer formed by a slot coat application.

18. The absorbent core of claim 16, wherein the base layer comprises an extrudate layer formed by a spray application.

19. The absorbent core of claim 1, wherein the three-dimensional extrudate layer is comprised of a network of extrudate filaments.

20. The absorbent core of claim 1, wherein the three-dimensional extrudate layer is comprised of a substantially continuous layer of extrudate, having voids therein.

21. The absorbent core of claim 1, wherein the three-dimensional extrudate layer has a height of about 100 to about 3000 μ.

22. The absorbent core of claim 1, wherein the three-dimensional extrudate layer has an open area of about 50% to about 99%.

23. The absorbent core of claim 1, wherein the three-dimensional extrudate layer is comprised of a material selected from the group consisting of: a hot-melt adhesive, a swelling adhesive, a wax, a thermoplastic, and mixtures and combinations thereof.

24. An absorbent article comprising:
a liquid pervious top sheet,
a liquid impervious back sheet, and
an absorbent core at least partially disposed between the top sheet and back sheet;
wherein the absorbent core comprises:
a central fibrous layer comprising synthetic fibers,
a three dimensional extrudate layer having a plurality of voids and being at least partially disposed beneath the central fibrous layer,
a base layer, and
superabsorbent polymer;
wherein the three-dimensional extrudate layer and base layer form a plurality of discrete containment wells in which at least some of the superabsorbent polymer is contained.

25. The absorbent article of claim 24, whereby the article has a first waist region, a second waist region longitudinally opposed to the first waist region, and a crotch region between the first and second waist regions, the article further comprising at least one fastening element attached to a lateral edge of the first waist region; and
one or more target devices attached to the article in the second waist region, where at least one fastening element and the one or more target devices are capable of attaching to one another, the one or more target devices being located so that the first waist region and second waist region of the garment may be joined to one another to secure the garment on a wearer.

26. The absorbent article of claim 25, further comprising elastic leg gathers comprising one or more elastic materials disposed adjacent a lateral edge of the crotch region, and standing leg gathers disposed on the top sheet adjacent the lateral edge of the crotch region.

27. The absorbent article of claim 25, wherein the at least one fastening element comprises a hook portion of a hook and loop fastener and the one or more target devices comprise the loop portion of a hook and loop fastener.

28. The absorbent article of claim 25, wherein the at least one fastening element is an adhesive tape and the one or more target devices comprise a tape receiving surface.

29. The absorbent article of claim 25, wherein the at least one fastening element is comprised of a pair of laterally extending tabs disposed on the lateral edges of the first waist region, whereby the laterally extending tabs each include at least one fastening element.

30. The absorbent article of claim 24, where the plurality of discrete containment wells further comprise at least a portion of the central fibrous layer.

31. The absorbent article of claim 24, where the plurality of discrete containment wells further comprise a second base layer.

32. The absorbent article of claim 24, where the plurality of discrete containment wells further comprise a layer selected from the group consisting of: a tissue layer, a non-woven layer, a back sheet layer, a wicking layer, a fluid transfer layer, a fluid handling layer, a storage layer, a fluid distribution layer, and combinations and fragments thereof.

33. The absorbent article of claim 24, where the synthetic fibers are selected from the group consisting of cellulose ester fibers, cellulose acetate fibers, rayon fibers, lyocell fibers, polyacrylonitrile fibers, polyester fibers, polypropylene fibers, polyethylene fibers, and mixtures and combinations thereof.

34. The absorbent article of claim 33, where the synthetic fibers are a cellulose ester fibers.

35. The absorbent article of claim 33,, where the synthetic fibers are cellulose acetate fibers.

36. The absorbent article of claim 33, where the synthetic fibers are polypropylene fibers.

37. The absorbent article of claim 24, where the synthetic fibers are substantially continuous. fibers.

38. The absorbent article of claim 24, where the synthetic fibers are tow fibers.

39. The absorbent article of claim 37, where the length of the synthetic fibers is substantially equal to the length of the absorbent core.

40. The absorbent article of claim 24, where the synthetic fibers are discontinuous fibers.

41. The absorbent article of claim 40, where the synthetic fibers are formed into a carded non-woven web.

42. The absorbent article of claim 24, wherein the absorbent core further comprises at least one additional layer.

43. The absorbent article of claim 42, where the at least one additional layer is selected from the group consisting of: a fluid transfer layer, a fluid handling layer, a storage layer, a wicking layer, a fluid distribution layer, and combinations and fragments thereof.

44. The absorbent article of claim 24, wherein the base layer comprises a materials selected from the group consisting of: an extrudate, a tissue layer, a nonwoven layer, a film layer, and combinations and fragments thereof.

45. The absorbent article of claim 44, wherein the base layer comprises an extrudate layer formed by a slot coat application.

46. The absorbent article of claim 44, wherein the base layer comprises an extrudate layer formed by a spray application.

47. The absorbent article of claim 24, wherein the three-dimensional extrudate layer is comprised of a network of extrudate filaments.

48. The absorbent article of claim 24, wherein the three-dimensional extrudate layer is comprised of a substantially continuous layer of extrudate, having voids therein.

49. The absorbent article of claim 24, wherein the three-dimensional extrudate layer has a height of about 100 μ to about 3000 μ.

50. The absorbent article of claim 24, wherein the three-dimensional extrudate layer has an open area of about 50% to about 99%.

51. The absorbent article of claim 24, wherein the three-dimensional extrudate layer is comprised of a material selected from the group consisting of: a hot-melt adhesive, a swelling adhesive, a wax, a thermoplastic, and mixtures and combinations thereof.

52. A method of making an absorbent article comprising:
a) preparing a top sheet and a back sheet;
b) preparing an absorbent core by:
b1) providing an central fibrous layer that comprises synthetic fibers;
b2) providing a three-dimensional extrudate layer having a plurality of voids;
b3) providing a base layer;
b4) disposing the base layer adjacent to the three-dimensional extrudate layer, whereby the base layer and the extrudate layer form a plurality of discrete containment wells;
b5) providing superabsorbent particles, whereby at least some of the superabsorbent particles are disposed in the containment wells; and
b6) disposing the central fibrous layer at least partially above the three-dimensional extrudate layer and the base layer; and
c) disposing the absorbent core at least partially between the top sheet and the back sheet.

53. The method of claim 52, wherein preparing the absorbent core further comprises attaching the central fibrous layer to the surface of the three-dimensional layer opposite the base layer, and
wherein the three-dimensional layer, the base layer and the central fibrous layer substantially enclose the superabsorbent particles within the plurality of containment wells.

54. The method of claim 52, wherein preparing the absorbent core further comprises providing another layer, wherein the other layer is disposed on the surface of the three-dimensional layer opposite the base layer, and
wherein the three-dimensional layer, the base layer and the other layer substantially enclose the superabsorbent particles within the plurality of containment wells.

55. The method of claim 54, whereby the other layer is selected from the group consisting of: an additional layer, a base layer, a fibrous layer, a tissue layer, a non-woven layer, a back sheet layer, a wicking layer, a fluid transfer layer, a fluid handling layer, a storage layer, a fluid distribution layer, and combinations and fragments thereof.

56. The method of claim 52, where the synthetic fibers are selected from the group consisting of cellulose ester fibers, cellulose acetate fibers, rayon fibers, lyocell fibers, polyacrylonitrile fibers, polyester fibers, polypropylene fibers, polyethylene fibers, and mixtures and combinations thereof.

57. The method of claim 56, where the synthetic fibers are a cellulose ester fibers.

58. The method of claim 57, where the synthetic fibers are cellulose acetate fibers.

59. The method of claim 56, where the synthetic fibers are polypropylene fibers.

60. The method of claim 52, where the synthetic fibers are substantially continuous fibers.

61. The method of claim 52, where the synthetic fibers are tow fibers.

62. The method of claim 60, where the length of the synthetic fibers is substantially equal to the length of the absorbent core.

63. The method of claim 52, where the synthetic fibers are discontinuous fibers.

64. The method of claim 63, where the synthetic fibers are formed into a carded non-woven web.

65. The method of claim 52, wherein providing the base layer comprises providing a roll-good material selected from the group consisting of: a tissue layer, a nonwoven layer, a film layer, and combinations and fragments thereof.

66. The method of claim 52, wherein providing the base layer comprises providing a substantially continuous extrudate layer.

67. The method of claim 66, wherein providing the base layer comprises extruding a substantially continuous film of extrudate from a continuous slot coat applicator.

68. The method of claim 66, wherein providing the base layer comprises extruding a substantially continuous extrudate layer from a spray applicator.

69. The method of claim 52, wherein providing the three-dimensional extrudate layer comprises a method selected from the group consisting of: hot melt spraying, bead extrusion, thin film extrusion, gravure printing, screen printing, transfer coating and combinations thereof.

70. The method of claim 52, wherein providing the three-dimensional extrudate layer comprises providing a network of extrudate filaments.

71. The method of claim 52, wherein providing the three-dimensional extrudate layer comprises providing a substantially continuous film of extrudate, having voids therein.

72. The method of claim 52, wherein the three-dimensional extrudate layer has a height of about 100 μ to about 3000 μ.

73. The method of claim 52, wherein the three-dimensional extrudate layer has an open area of about 50% to about 99%.

74. The method of claim 52, wherein the three-dimensional extrudate layer is comprised of a material selected from the group consisting of: a hot-melt adhesive, a swelling adhesive, a wax, a thermoplastic, and mixtures and combinations thereof.

75. The method of claim 52, wherein providing the absorbent core further comprises extruding the three-dimensional extrudate layer directly onto the base layer.

76. The method of claim 52, wherein providing the absorbent core further comprises disposing the three-dimensional layer on the base layer such that the two layers are in intimate contact.

77. The method of claim 52, wherein providing the absorbent core further comprises providing at least one additional layer.

78. The method of claim 77, where the at least one additional layer is selected from the group consisting of: a fluid transfer layer, a fluid handling layer, a storage layer, a wicking layer, a fluid distribution layer, and combinations and fragments thereof.

79. The method of claim 77, wherein the at least one additional layer is disposed between two layers of the absorbent core.

80. The method of claim 52, further comprising providing at least one additional layer above or below the absorbent core.

81. The method of claim 80, where the at least one additional layer is selected from the group consisting of: a fluid transfer layer, a fluid handling layer, a storage layer, a wicking layer, a fluid distribution layer, and combinations and fragments thereof.

82. The method of claim 52, further comprising working the absorbent core using a mechanical and/or thermal process.

83. The method of claim 82, further comprising working the absorbent core with the top sheet.

84. The method of claim 82, further comprising working the absorbent core with the back sheet.
